(19) United States
(12) Patent Application Publication
(10) Pub. No.: US 2008/0019934 A1
(43) Pub. Date: Jan. 24, 2008

(54) Water-in-oil emusions for the treatment of keratin fibers, comprising at least one cyanoacrylate monomer and aqueous ammonia

(76) Inventors: Henri Samain, Bievres (FR); Leila Hercouet, Neuilly Plaisance (FR); Geraldine Fack, Levallois (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/783,929

(22) Filed: Apr. 13, 2007

Related U.S. Application Data
(60) Provisional application No. 60/796,533, filed on May 2, 2006.

(30) Foreign Application Priority Data
Apr. 13, 2006 (FR) 0603319

(51) Int. Cl. A61K 8/25; A61Q 5/00

(52) U.S. Cl. 424/70.11; 424/70.1

(57) ABSTRACT

The present disclosure relates to a water-in-oil emulsion which comprises an aqueous phase comprising at least one surfactant; at least one nitrogenous alkaline agent of the aqueous ammonia or amine type and an oily phase comprising at least one cyanoacrylate monomer. The water-in-oil emulsion of the present disclosure can make it possible to reduce ammonia odors during use.

This application claims benefit of U.S. Provisional Application No. 60/796,533, filed May 2, 2006, the contents of which are incorporated herein by reference. This application also claims benefit of priority under 35 U.S.C. § 119 to French Patent Application No. FR 06/03319, filed Apr. 13, 2006, the contents of which are also incorporated herein by reference.

The present disclosure relates to a water-in-oil emulsion for the treatment of keratin fibers, such as the hair, comprising at least one cyanoacrylate monomer.

It is known practice to dye keratin fibers using dye precursors in order to obtain permanent colorations. This type of coloration may require the use of aqueous ammonia in order to promote the penetration of the dye precursors into the keratin fiber and the oxidative reaction responsible for the appearance of the color within this fiber.

The use of these aqueous-ammonia-based compositions can have an unpleasant side-effect because of the odor due to the presence of aqueous ammonia. The higher the pH of the composition, the stronger the odor of these compositions. The same unpleasantness is present for other types of hair treatments, such as permanent-waving, hair straightening or bleaching, and also for other compositions in which aqueous ammonia has been replaced with other nitrogenous alkaline agents such as ethanolamine, and the like.

Thus, there is a need in the art for compositions based on aqueous ammonia or on any other nitrogenous alkaline compound which are just as effective as the existing compositions, but without the unpleasant aspects associated with the odor during use.

The present disclosure can fill this need. Accordingly, the present disclosure relates to a water-in-oil emulsion which comprises an aqueous phase comprising at least one surfactant; at least one nitrogenous alkaline agent of the aqueous ammonia or amine type and an oily phase comprising at least one cyanoacrylate monomer that can polymerize anionically in the presence of at least one nucleophilic agent.

The water-in-oil emulsion of the present disclosure can make it possible to considerably reduce the typical odor of ammonia during use on keratin fibers.

The subject of the present disclosure is also a process for treating keratin fibers which comprises the application, to the keratin fibers, of the emulsion of the disclosure and also the use of this emulsion for the treatment of keratin fibers such as the hair.

The emulsion of the disclosure comprises an oily phase. This oily phase can comprise at least one organic solvent. The organic solvents can be chosen from compounds that are liquid at a temperature of 25° C. and under 10⁵ Pa (760 mm Hg).

In the context of the disclosure, the at least one cyanoacrylate monomer and the at least one organic solvent are distinct. The oily phase may comprise the at least one cyanoacrylate monomer (which is an electrophilic monomer).

The at least one organic solvent can be, by way of non-limiting example, chosen from:
aromatic alcohols such as benzyl alcohol;
liquid fatty alcohols such as C₁₀-C₃₀ fatty alcohols;
modified and unmodified polyols such as glycerol, glycol, propylene glycol, dipropylene glycol, butylene glycol and butyl diglycol;
volatile silicones such as cyclopentasiloxane, cyclohexasiloxane, polydimethylsiloxanes which may or may not be modified with alkyl and/or amine and/or imine and/or fluoroalkyl and/or carboxylic and/or betaine and/or quaternary ammonium functional groups, liquid modified polydimethylsiloxanes, mineral, organic and plant oils, alkanes, and for example, C₅ to C₁₀ alkanes;
liquid fatty acids, liquid fatty esters, and for example, liquid fatty alcohol benzoates and salicylates.

The at least one organic solvent is, in at least one embodiment, chosen from organic oils, for example:
silicones such as volatile silicones, amino and non-amino silicone gums and oils, and mixtures thereof;
mineral oils;
plant oils such as olive oil, castor oil, rapeseed oil, coconut oil, wheatgerm oil, sweet almond oil, avocado oil, macadamia oil, apricot oil, safflower oil, candlenut oil, camelina oil, tamanu oil, lemon oil, and alternatively organic compounds such as C₅-C₁₀ alkanes, acetone, methyl ethyl ketone, esters of liquid C₁-C₂₀ acids and of C₁-C₈ alcohols such as methyl acetate, butyl acetate, ethyl acetate and isopropyl myristate, dimethoxyethane, diethoxyethane, liquid C₁₀-C₃₀ fatty alcohols such as oleyl alcohol, esters of liquid C₁₀-C₃₀ fatty alcohols such as C₁₀-C₃₀ fatty alcohol benzoates, and mixtures thereof; and
polybutene oil, isononyl isononanoate, isostearyl malate, pentaerythrityl tetraisostearate, tridecyl trimellitate, and the mixture of cyclopentasiloxane (14.7% by weight)/polydimethylsiloxane dihydroxylated in the α and ω positions (85.3% by weight), and mixtures thereof.

According to at least one embodiment, the at least one organic solvent comprises at least one silicone such as liquid polydimethylsiloxanes and modified liquid polydimethylsiloxanes, their viscosity at 25° C. being in a range from 0.1 cst to 1,000,000 cst, for example, from 1 cst to 30,000 cst.

In at least one embodiment, the oily phase is chosen from oils and mixtures thereof, for example:
the mixture of α,ω-dihydroxylated polydimethyl siloxane/cyclopentadimethylsiloxane (14.7/85.3) sold by Dow Corning under the name DC 1501 Fluid;
the mixture of α,ω-dihydroxylated poly dimethylsiloxane/polydimethylsiloxane sold by Dow Corning under the name DC 1503 Fluid;
the mixture of dimethicone/cyclopentadimethylsiloxane sold by Dow Corning under the name DC 1411 Fluid, or that sold by Bayer under the name SF1214;
the cyclopentadimethylsiloxane sold by Dow Corning under the name DC245 Fluid;
and the respective mixtures of these oils.

According to at least one embodiment of the disclosure, the oily phase is chosen from volatile silicones, alone and as a mixture with another silicone.

The volatile silicones that can be used in the disclosure may be chosen from linear and cyclic silicones which have a viscosity at ambient temperature and under atmospheric pressure of less than 8 mm²/s (8 cSt).

The viscosity is, in at least one embodiment, measured by capillary viscometry, for example using a capillary viscometer, such as a Ubbelohde type, at a temperature of 25° C., according to ASTM standard D445-97. The “falling-ball” method may also be used.

The volatile silicones may have a boiling point in a range from 60° C. to 260° C., and, in at least one embodiment, are chosen from:
(i) cyclic volatile silicones containing from 3 to 7, for example, from 4 to 5, silicon atoms. They are, for example, octamethylcyclotetrasiloxane sold under the name “Volatile Silicone 7207” by Union Carbide or “Silbione® 70045 V 2” by Rhodia, decamethylcyclopentasiloxane, commonly called D5, sold under the name “Volatile Silicone 7158” by Union Carbide, “Silbione® 70045 V 5” by Rhodia, or under the name DC245 Fluid by Dow Corning, and mixtures thereof.

Non-limiting mention may also be made of cyclocopolymers of the dimethylsiloxane/methylalkylsiloxane type, such as “Silicone Volatile FZ 3109” sold by the company Union Carbide, having the chemical structure:

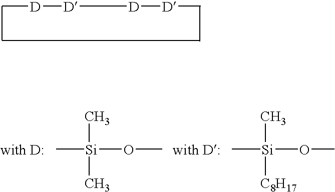

Further non-limiting mention may also be made of mixtures of cyclic silicones with organosilicon compounds, such as the mixture of octamethylcyclotetrasiloxane and tetratrimethylsilylpentaerythritol (50/50) and the mixture of octamethylcyclotetrasiloxane and oxy-1,1′-bis-[2,2,2′,2′,3,3′-hexakis(trimethylsilyloxy)]neopentane;
(ii) linear volatile silicones containing from 2 to 9 silicon atoms and having a viscosity of less than or equal to 5 mm²/s at 25° C. An example is decamethyltetrasiloxane sold, for example, under the name “SH 200” by the company Toray Silicone. Silicones belonging to this category are also described in the article published in Cosmetics and Toiletries, Vol. 91, Jan. 76, p. 27-32—Todd & Byers “Volatile Silicone fluids for cosmetics.”

The oily phase may be included in the emulsion in an amount ranging from 0.1% to 50% by weight of the emulsion, such as from 1% to 30% by weight of emulsion, and for example, from 5% to 25%.

When the oily phase comprises at least one volatile silicone, the at least one silicone may be present in an amount ranging from 0.1% to 30% by weight, such as from 5% to 20% by weight, and for example, from 8% to 15% by weight relative to the total weight of emulsion.

The at least one surfactant that can be used in the emulsion of the disclosure may be chosen from any surfactant known in the art to form water-in-oil emulsions. The surfactant may be anionic, amphoteric, cationic or non-ionic.

Non-limiting examples of the at least one surfactant that can be used in the disclosure, include non-silicone anionic surfactants, for example, the salts of the following compounds: alkyl sulphates, alkyl ether sulphates, alkylamido ether sulphates, alkylarylpolyether sulphates, monoglyceride sulphates; alkyl sulphonates, alkyl phosphates, alkylamide sulphonates, alkylaryl sulphonates, α-olefin sulphonates, paraffin sulphonates; alkyl sulphosuccinates, alkyl ether sulphosuccinates, alkylamide sulphosuccinates, alkyl sulphosuccinimates, alkyl sulphoacetates, alkyl ether phosphates, acyl sarcosinates, acyl isethionates and N-acyltaurates, the alkyl or acyl radicals of these listed compounds, for example, containing from 12 to 20 carbon atoms, and the aryl radicals of these listed compounds, for example, a phenyl or benzyl group. The salts of these compounds are, for example, alkali metal salts, such as sodium salts, ammonium salts, amine salts, amino alcohol salts or magnesium salts. In at least one embodiment, the at least one surfactant is chosen from anionic surfactants, for example, the salts of fatty acids, such as the salts of oleic acid, ricinoleic acid, palmitic acid or stearic acid, coconut oil acid or hydrogenated coconut oil acid; and acyl lactylates wherein the acyl radical contains 8 to 20 carbon atoms. Non-limiting examples of the at least one surfactant include weakly anionic surfactants, such as alkyl-D-galactosiduronic acids and their salts, and also polyoxyalkylenated ether carboxylic acids and their salts, for example, those comprising from 2 to 50 ethylene oxide groups, and mixtures thereof. The anionic surfactants of the polyoxyalkylenated ether carboxylic salt or acid type can be, for example, those of formula (I):
R₁—(OC₂H₄)ₙ—OCH₂COOA (1)
wherein:

R1 is chosen from alkyl and alkylaryl groups, and n is an integer or decimal number (average value) that can range from 2 to 24, for example, from 3 to 10, the alkyl radical having from 6 to 20 carbon atoms, and the aryl, in at least one embodiment, is phenyl.

A is chosen from a hydrogen atom, ammonium, sodium, potassium, lithium and magnesium ions and monoethanolamine and triethanolamine residues. The at least one surfactant may also be chosen from mixtures of compounds of formula (I), for example, mixtures wherein the groups R1 are different.

In at least one embodiment, the at least one surfactant is chosen from anionic surfactants, such as alkyl sulphate salts and alkyl ether sulphate salts, and mixtures thereof.

According to at least one embodiment, the at least one surfactant is chosen from non-silicone amphoteric surfactants, such as secondary and tertiary aliphatic amine derivatives, wherein the aliphatic radical is chosen from linear and branched chains containing from 8 to 22 carbon atoms and containing at least one water-soluble anionic group, for example carboxylate, sulphonate, sulphate, phosphate or phosphonate; in at least one further embodiment, the at least one surfactant is chosen from (C₈-C₂₀)alkylbetaines, sulphobetaines, (C₈-C₂₀)alkylamido(C₁-C₆)alkylbetaines and (C₈-C₂₀)alkylamido(C₁-C₆)alkylsulphobetaines.

According to at least one embodiment, the at least one surfactant is chosen from amine derivatives, for example, products sold under the name Miranol, as described in U.S. Pat. Nos. 2,528,378 and 2,781,354 and having the structures:
R2-CONHCH2CH2-N(R3)(R4)(CH2COO—) (2)
wherein:

R2 is chosen from alkyl radicals derived from an acid R2-COOH present in hydrolysed coconut oil, heptyl radicals, nonyl radicals and undecyl radicals, R3 is a α-hydroxyethyl group and R4 is a carboxymethyl group; and
R5-CONHCH2CH2-N(B)(C) (3)
wherein:

B is —CH₂CH₂OX′, C is —(CH₂)_{z}—Y′, wherein z=1-2,

X′ is chosen from —CH₂CH₂—COOH and a hydrogen atom,

Y′ is chosen from —COOH and —CH₂—CHOH—SO₃H, and R5 is chosen from alkyl radicals of an acid R5-COOH present in hydrolyzed linseed oil or coconut oil, alkyl radicals, for example, C₇, C₉, C₁₁ and C₁₃ alkyl radicals, C₁₋₇ alkyl radicals and their iso form, and unsaturated C₁₇ radicals.

Such compounds are classified in the CTFA dictionary, 5th edition, 1993, under the names Disodium Cocoamphodiacetate, Disodium Lauroamphodiacetate, Disodium Caprylamphodiacetate, Disodium Capryloamphodiacetate, Disodium Cocoamphodipropionate, Disodium Lauroamphodipropionate, Disodium Caprylamphodipropionate, Disodium Capryloamphodipropionate, Lauroamphodipropionic acid, and Cocoamphodipropionic acid.

In at least one embodiment, the at least one surfactant is chosen from the cocoamphodiacetate sold under the trade name Miranol C2M concentrate by the company Rhone Poulenc.

Non-limiting examples of the at least one surfactant include non-silicone non-ionic surfactants, such as the non-ionic surfactants that are well known per se (see, for example, in this regard “Handbook of Surfactants” by M. R. Porter, publishers Blackie & Son (Glasgow and London), 1991, pp 116-178). Further non-limiting examples include alcohols, alpha-diols, alkylphenols or polyethoxylated, polypropoxylated and polyglycerolated fatty acids having a fatty chain containing, for example, from 8 to 18 carbon atoms, it being possible for the number of ethylene oxide or propylene oxide groups to range, for example, from 2 to 50 and for the number of glycerol groups to range, for example, from 2 to 30. Other non-limiting examples include copolymers of ethylene oxide and of propylene oxide, condensates of ethylene oxide and of propylene oxide with fatty alcohols; polyethoxylated fatty amides, for example, having from 2 to 30 mol of ethylene oxide, polyglcyerolated fatty amides comprising, on average, from 1 to 5 glycerol groups, such as from 1.5 to 4; polyethoxylated fatty amines, for example, having 2 to 30 mol of ethylene oxide; oxyethylenated sorbitan fatty acid esters having from 2 to 30 mol of ethylene oxide; sucrose fatty acid esters, polyethylene glycol fatty acid esters, alkylpolyglycosides, and N-alkylglucamine derivatives, amine oxides such as (C₁₀C₁₄)alkylamine oxides and N-acylaminopropylmorpholine oxides. According to one embodiment of the disclosure, the at least one surfactant is chosen from non-ionic surfactants such as alkylpolyglycosides.

In at least one embodiment, the at least one surfactant is chosen from non-silicone cationic surfactants, for example, surfactants that are well known per se, such as salts of primary, secondary and tertiary fatty amines, which are optionally polyoxyalkylenated, quaternary ammonium salts, and mixtures thereof.

In at least one further embodiment, the at least one surfactant is chosen from quaternary ammonium salts, for example:
those of formula (VI): 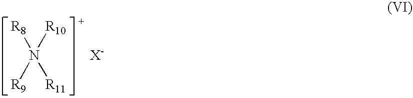
   wherein:

R₈ to R₁₁, which may be identical or different, are chosen from linear and branched aliphatic radicals containing from 1 to 30 carbon atoms, and aromatic radicals such as aryl and alkylaryl. The aliphatic radicals can contain heteroatoms such as oxygen, nitrogen, sulphur and halogens. The aliphatic radicals are, for example, chosen from alkyl, alkoxy, (C₂-C₆)polyoxyalkylene, alkylamide, (C₁₂-C₂₂)alkylamido(C₂-C₆)alkyl, (C₁₂-C₂₂)alkyl acetate, and hydroxyalkyl radicals, containing from 1 to 30 carbon atoms;

X is an anion chosen from halides, phosphates, acetates, lactates, (C₂-C₆)alkyl sulphates, alkyl sulphonates and alkylaryl sulphonates;
quaternary ammonium salts of imidazoline, for example, those of formula (VII): 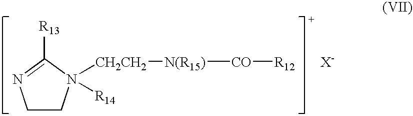
   wherein:

R12 is chosen from alkenyl and alkyl radicals containing from 8 to 30 carbon atoms, for example fatty acid derivatives of tallow, R13 is chosen from a hydrogen atom, C₁-C₄ alkyl radicals and alkenyl and alkyl radicals containing from 8 to 30 carbon atoms, R14 is chosen from C₁-C₄ alkyl radicals, R15 is chosen from a hydrogen atom and C₁-C₄ alkyl radicals, X— is an anion chosen from halides, phosphates, acetates, lactates, alkyl sulphates, alkyl sulphonates and alkylaryl sulphonates. In at least one embodiment, R12 and R13 are chosen from a mixture of alkenyl and/or alkyl radicals containing from 12 to 21 carbon atoms, for example fatty acid derivatives of tallow, R14 is a methyl radical, and R15 is a hydrogen atom. Such a product is, for example, sold under the name Rewoquat® W 75 by the company Rewo;
the diquaternary ammonium salts of formula (VIII): 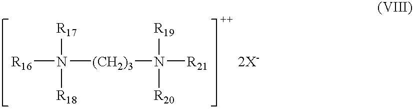
   wherein:

R16 is chosen from aliphatic radicals containing from 16 to 30 carbon atoms, R17, R18, R19, R20 and R21, which may be identical or different, are chosen from hydrogen atoms and alkyl radicals containing from 1 to 4 carbon atoms, and X is an anion chosen from halides, acetates, phosphates, nitrates and methyl sulphates. Such diquaternary ammonium salts comprise, for example, propanetallowediammonium dichloride;
quaternary ammonium salts containing at least one ester functional group, such as those of formula (IX): 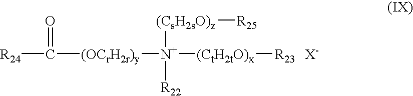
   wherein:

R22 is chosen from C₁-C₆ alkyl radicals and C₁-C₆ hydroxyalkyl and dihydroxyalkyl radicals;

R23 is chosen from:
the radical 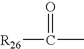
linear and branched, saturated and unsaturated C₁-C₂₂ hydrocarbon-based radicals R27, and
a hydrogen atom,

R25 is chosen from:
the radical 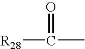
linear and branched, saturated and unsaturated C₁-C₆ hydrocarbon-based radicals R29, and
a hydrogen atom,

R24, R26 and R28, which may be identical or different, are chosen from linear and branched, saturated and unsaturated C₇-C₂₁ hydrocarbon-based radicals;

r, s and t, which may be identical or different, are integers ranging from 2 to 6;

y is an integer ranging from 1 to 10;

x and z, which may be identical or different, are integers ranging from 0 to 10;

X— is chosen from simple and complex, organic and inorganic anions; with the proviso that the sum x+y+z is from 1 to 15, that when x is 0, then R23 is R27 and that, when z is 0, then R25 is R29.

In at least one embodiment, R22 is chosen from linear and branched alkyl radicals, for example, linear.

In at least one further embodiment, R22 is chosen from methyl, ethyl, hydroxyethyl and dihydroxypropyl radicals, for example, methyl and ethyl radicals.

According to at least one embodiment, the sum x+y+z is from 1 to 10.

In at least one embodiment, R23 is chosen from hydrocarbon-based radical R27, for example, R27 may be long and have from 12 to 22 carbon atoms, or for example, R27 may be short and have from 1 to 3 carbon atoms.

In at least one further embodiment, R25 is chosen from hydrocarbon-based radical R29, for example, R29 having from 1 to 3 carbon atoms.

According to at least one embodiment, R24, R26 and R28, which may be identical or different, are chosen from linear and branched, saturated and unsaturated C₁₁-C₂₁ hydrocarbon-based radicals, for example, from linear and branched, saturated and unsaturated C₁₁-C₂₁ alkyl and alkenyl radicals.

In at least one embodiment, x and z, which may be identical or different, are chosen from 0 and 1.

According to at least one embodiment, y is equal to 1.

In at least one further embodiment, r, s and t, which may be identical or different, are chosen from 2 and 3, for example, 2.

According to at least one embodiment, X— is chosen from halides (for example, chloride, bromide and iodide) and alkyl sulphates, for example, methyl sulphate. In at least one further embodiment, X— is chosen from methanesulphonate, phosphate, nitrate, tosylate, anions derived from organic acids such as acetate or lactate, and any other anion compatible with the ammonium containing an ester functional group.

In at least one embodiment, X— is chosen from chloride and methyl sulphate.

According to at least one embodiment, the at least one surfactant is chosen from the ammonium salts of formula (IX) wherein:
R22 is chosen from methyl and ethyl radicals;
x and y are equal to 1;
z is chosen from 0 and 1;
r, s and t are equal to 2;
R₂₃ is chosen from:
   the radical 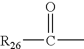
   methyl, ethyl and C₁₄-C₂₂ hydrocarbon-based radicals, and
   a hydrogen atom;
R25 is chosen from:
   the radical 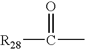
   and
   a hydrogen atom;
R24, R26 and R28, which may be identical or different, are chosen from linear and branched, saturated and unsaturated C₁₃-C₁₇ hydrocarbon-based radicals, for example, linear and branched, saturated and unsaturated C₁₃-C₁₇ alkyl and alkenyl radicals.

For example, in at least one embodiment, the hydrocarbon-based radicals mentioned above are linear.

In at least one embodiment, the at least one surfactant may be chosen from compounds of formula (IX) such as diacyloxyethyldimethylammonium, diacyloxyethylhydroxyethylmethylammonium, monoacyloxyethyldihydroxyethylmethylammonium, triacyloxyethylmethylammonium and monoacyloxyethylhydroxyethyldimethylammonium salts (for example, chloride and methyl sulphate), and mixtures thereof. The acyl radicals, for example, have 14 to 18 carbon atoms and originate, for example, from a plant oil such as palm oil or sunflower oil. When the compound contains several acyl radicals, the latter may be identical or different.

These products can be obtained, for example, by direct esterification of optionally oxyalkylenated triethanolamine, triisopropanolamine, alkyldiethanolamine or alkyldiisopropanolamine with fatty acids or with mixtures of fatty acids of plant or animal origin, or by transesterification of their methyl esters. This esterification may be followed by a quaternization using an alkylating agent such as an alkyl (for example, methyl or ethyl) halide, a dialkyl (for example, dimethyl or diethyl) sulphate, methyl methanesulphonate, methyl para-toluenesulphonate, and glycol or glycerol chlorohydrin.

Such compounds are, for example, sold under the names Dehyquart® by the company Henkel, Stepanquat® by the company Stepan, Noxamium® by the company Ceca, and Rewoquat® WE 18 by the company Rewo-Witco.

In at least one embodiment, the at least one surfactant is chosen from cationic surfactants, for example, a mixture of quaternary ammonium salts of mono-, di- and triesters with a weight majority of diester salts can be used.

Mixtures of ammonium salts that may be used include, for example, the mixture comprising from 15% to 30% by weight of acyloxyethyldihydroxyethylmethylammonium methyl sulphate, from 45% to 60% of diacyloxyethylhydroxyethylmethylammonium methyl sulphate and from 15% to 30% of triacyloxyethylmethylammonium methyl sulphate, the acyl radicals having from 14 to 18 carbon atoms and being derived from palm oil that is optionally partially hydrogenated.

In at least one embodiment, the at least one surfactant is chosen from ammonium salts comprising at least one ester functional group that are described in U.S. Pat. Nos. 4,874,554 and 4,137,180.

According to at least one embodiment, the at least one surfactant is chosen from quaternary ammonium salts of formula (VI), for example, firstly, tetraalkylammonium chlorides such as dialkyldimethylammonium chlorides and alkyltrimethylammonium chlorides, wherein the alkyl radical contains from 12 to 22 carbon atoms, for example, behenyltrimethylammonium chloride, distearyldimethylammonium chloride, cetyltrimethylammonium chloride and benzyldimethylstearylammonium chloride, and, secondly, palmitylamidopropyltrimethylammonium chloride and stearamidopropyldimethyl(myristyl acetate)ammonium chloride sold under the name Ceraphyl® 70 by the company Van Dyk.

In at least one further embodiment, the at least one surfactant is chosen from cationic surfactants chosen from quaternary ammonium salts, for example, behenyltrimethylammonium chloride and palmitylamidopropyltrimethylammonium chloride.

The non-silicone surfactants mentioned above can be present in the emulsion of the present disclosure in an amount ranging from 0.1% to 30% by weight of active material; for example, from 0.5% to 15% by weight of composition.

In at least one embodiment, the at least one surfactant is chosen from silicone surfactants, for example those described in French Patent No. FR2 818 902. The silicone surfactants that can be used in the present disclosure are those which are well known to those skilled in the art. They may be water-soluble, spontaneously water-dispersible or water-insoluble. In at least one further embodiment, the at least one surfactant is chosen from silicone surfactants that are water-soluble and spontaneously water-dispersible.

According to at least one embodiment, the at least one surfactant is chosen from silicone surfactants, for example, chosen from the compounds of the general formulae below: 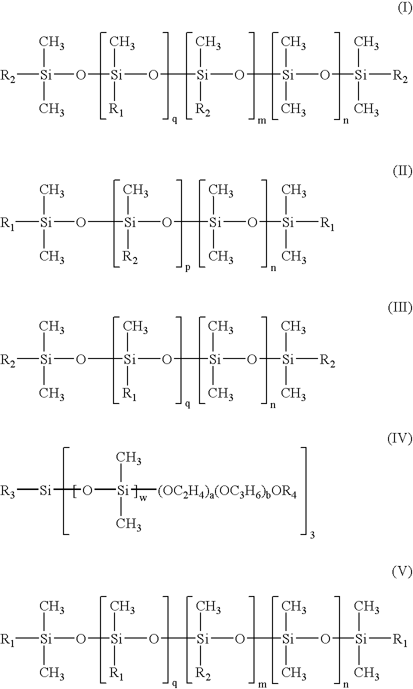
wherein:
R1, which may be identical or different, is chosen from linear and branched C₁-C₃₀ alkyl groups and a phenyl group;
R2, which may be identical or different, is chosen from —C_{c}H_{2c}—O—(C₂H₄O)ₐ—(C₃H₆O)_{b}—R5 and —C_{c}H_{2c}—O—(C₄H₈O)ₐ—R5;
R3 and R4, which may be identical or different, are chosen from linear and branched C₁-C₁₂ alkyl groups, for example, a methyl group;
R5, which may be identical or different, is chosen from a hydrogen atom, linear and branched alkyl groups containing from 1 to 12 carbon atoms, linear and branched alkoxy groups containing from 1 to 6 carbon atoms, linear and branched acyl groups containing from 2 to 12 carbon atoms, a hydroxyl group, —SO₃M, —OCOR6, C₁-C₆ aminoalkoxy groups optionally substituted on the amine, C₂-C₆ aminoacyl groups optionally substituted on the amine, —NHCH₂CH₂COOM, —N(CH₂CH₂COOM)₂, C₁-C₁₂ aminoalkyl groups optionally substituted on the amine and on the alkyl chain, C₁-C₃₀ carboxyacyl groups, phosphono groups optionally substituted with one or two substituted C₁-C₁₂ aminoalkyl groups, —CO(CH₂)_{d}COOM, —OCOCHR7(CH₂)_{d}COOM, —NHCO(CH₂)_{d}OH, and —NH₃Y;
M, which may be identical or different, is chosen from a hydrogen atom, Na, K, L₁, NH₄ and organic amines;
R6 is chosen from linear and branched C₁-C₃₀ alkyl groups,
R7 is chosen from a hydrogen atom and an SO₃M group;
d ranges from 1 to 10;
m ranges from 0 to 20;
n ranges from 0 to 500;
p ranges from 1 to 50;
q ranges from 0 to 20;
a ranges from 0 to 50;
b ranges from 0 to 50;
a+b is greater than or equal to 1;
c ranges from 0 to 4;
w ranges from 1 to 100;
Y is chosen from monovalent inorganic and organic anions such as halides (for example, chloride and bromide), a sulphate and a carboxylate (for example, acetate, lactate, and citrate).

In at least one embodiment, the at least one surfactant is chosen from silicone surfactants chosen from those of formulae (IV) and (VII) as defined above, for example, those of formulae (IV) and (VII) wherein at least one, and for example, all, of the following conditions are met:
c is in a range from 2 to 3;
R1 is a methyl group;
R5 is chosen from a hydrogen atom, a methyl group and an acetyl group, for example, a hydrogen atom;
a ranges from 1 to 25, for example, from 2 to 25;
b ranges from 0 to 25, for example, from 10 to 20;
n ranges from 0 to 100;
p ranges from 1 to 20.

In at least one further embodiment, the at least one surfactant is chosen from silicone surfactants, for example, those sold under the trade names Fluid DC 193 and DC 5225C by the company Dow Corning, Silwet® L 77 by the company OSI and Mazil® 756 by the company Mazer PPG, and the mixture of Lauryl PEG/PPG-18/18 Methicone (and) Poloxamer 407 (and) Dodecene sold by Dow Corning under the name DC 5200.

Although any type of ionicity can be used for the emulsions of the present disclosure, in at least one embodiment, the at least one surfactant is chosen from surfactants having an HLB (hydrophilic lipophilic balance) of less than 10. For example, the non-ionic surfactants that may be used in the compositions of the disclosure can have an HLB ranging from 1.5 to 10, such as from 1.5 to 7. The HLB or hydrophilic-lipophilic balance of the non-ionic surfactant(s) used according to the disclosure is the HLB according to Griffin defined in the publication J. Soc. Cosm. Chem. 1954 (Volume 5), pages 249-256.

The at least one surfactant may be present in an amount ranging from 0.01% to 30% by weight, such as from 0.1% to 30% by weight, and for example, from 0.2% to 15% by weight relative to the total weight of the emulsion.

In at least one embodiment, the at least one surfactant present in the emulsion is chosen from silicone surfactants.

The silicone surfactants may be present in the emulsion of the present disclosure in an amount ranging from 0.1% to 30% by weight of emulsion, for example, from 0.2% to 15% by weight of emulsion.

In at least one embodiment, the at least one cyanoacrylate monomer present in the composition of the disclosure is chosen from the monomers of formula (IX): 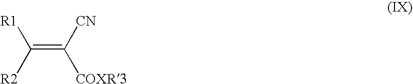
wherein:
X is chosen from NH, S and O,
R1 and R2 are chosen from, independently of one another, sparingly electron-withdrawing and non-electron-withdrawing groups (sparingly inductive-withdrawing and non-inductive-withdrawing groups) such as:
   hydrogen atoms,
   saturated and unsaturated, linear, branched and cyclic hydrocarbon-based groups, for example, containing from 1 to 20 carbon atoms, such as from 1 to 10 carbon atoms and optionally containing at least one entity chosen from nitrogen, oxygen and sulphur atom, and optionally substituted with at least one entity chosen from —OR, —COOR, —COR, —SH, —SR, —OH, and halogens,
   modified and unmodified polyorganosiloxane residues,
   polyoxyalkylene groups,
   R is chosen from saturated and unsaturated, linear, branched and cyclic hydrocarbon-based groups, for example, containing from 1 to 20 carbon atoms, such as from 1 to 10 carbon atoms and optionally containing at least one entity chosen from nitrogen, oxygen and sulphur atoms, and optionally substituted with at least one entity chosen from —OR′, —COOR′, —COR′, —SH, —SR′, —OH, and halogens, and polymer residues that may be obtained by free-radical polymerization, by polycondensation and by ring opening, R′ is chosen from C₁-C₁₀ alkyl groups, and
   R′3 is chosen from a hydrogen atom or R.

As disclosed herein, the terms “electron-withdrawing” and “inductive-withdrawing group (—I)” mean any group that is more electronegative than carbon. Reference may be made to the publication PR Wells Prog. Phys. Org. Chem., Vol 6, 111 (1968).

As disclosed herein, the terms “sparingly electron-withdrawing” and “non-electron-withdrawing group” mean any group whose electronegativity is less than or equal to that of carbon.

The alkenyl and/or alkynyl groups useful herein, in at least one embodiment, comprise from 2 to 20 carbon atoms, such as from 2 to 10 carbon atoms.

In at least one embodiment, the saturated and unsaturated, linear, branched and cyclic hydrocarbon-based groups mentioned above comprise, for example, from 1 to 20 carbon atoms and are chosen from cycloalkyl groups, aromatic groups and linear and branched alkyl, alkenyl or alkynyl groups, such as methyl, ethyl, n-butyl, tert-butyl, isobutyl, pentyl, hexyl, octyl, butenyl or butynyl.

In at least one further embodiment, substituted hydrocarbon-based groups useful herein are chosen from hydroxyalkyl and polyhaloalkyl groups.

Non-limiting examples of the unmodified polyorganosiloxanes mentioned above include polyalkylsiloxanes such as polydimethylsiloxanes, polyarylsiloxanes such as polyphenylsiloxanes, and polyarylalkylsiloxanes such as polymethylphenylsiloxanes.

Among the modified polyorganosiloxanes mentioned above, non-limiting mention may be made of polydimethylsiloxanes containing polyoxyalkylene and/or siloxy and/or silanol and/or amine and/or imine and/or fluoroalkyl groups.

Among the polyoxyalkylene groups disclosed above, non-limiting mention may be made of polyoxyethylene groups and polyoxypropylene groups, having, in at least one embodiment, from 1 to 200 oxyalkylene units.

Among the mono- and polyfluoroalkyl groups useful herein, non-limiting examples include —(CH₂)ₙ—(CF₂)ₘ—CF₃ and —(CH₂)ₙ—(CF₂)ₘ—CHF₂ wherein n=1 to 20 and m=1 to 20.

R1 and R2 may be optionally substituted with a group having a cosmetic activity. For example, groups having cosmetic activities include groups having coloring, antioxidant, UV-screening and/or conditioning functions.

Groups with a coloring function, may be chosen, for example, from azo, quinone, methine, cyanomethine and triarylmethane groups.

Groups with an antioxidant function, may be chosen, for example, from groups of butylhydroxyanisole (BHA), butylhydroxytoluene (BHT) and vitamin E type.

Groups with a UV-screening function, may be chosen, for example, from groups of benzophenone, cinnamate, benzoate, benzylidenecamphor and benzoylmethane type.

Groups with a conditioning function, may be chosen, for example, from cationic groups of fatty ester type.

In at least one embodiment, R1 and R2 are hydrogen atoms, and

R′3 is chosen from a hydrogen atom and saturated and unsaturated, linear, branched and cyclic hydrocarbon-based groups, for example, containing from 1 to 20 carbon atoms, such as from 1 to 10 carbon atoms and optionally containing at least one entity chosen from nitrogen, oxygen and sulphur atoms, and optionally substituted with at least one entity chosen from —OR′, —COOR′, —COR′, —SH, —SR′, —OH, and halogens, and polymer residues that may be obtained by free-radical polymerization, by polycondensation and by ring opening, R′ is chosen from C₁-C₁₀ alkyl groups.

According to at least one embodiment, R′3 is chosen from saturated hydrocarbon-based groups containing from 1 to 10 carbon atoms.

In at least one further embodiment, X is O.

In at least one embodiment, the at least one cyanoacrylate monomer is chosen from compounds of formula (IX), for example:

a) the family of polyfluoroalkyl 2-cyanoacrylates, such as: the ester 2,2,3,3-tetrafluoropropyl 2-cyano-2-propenoate of formula (X):

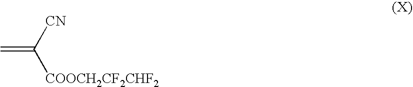

and the ester 2,2,2-trifluoroethyl 2-cyano-2-propenoate of formula (XI):

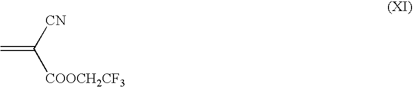

b) the alkyl and alkoxyalkyl 2-cyanoacrylates 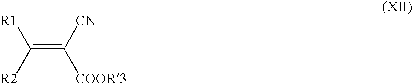
wherein:
R′3 is chosen from C₁-C₁₀ alkyl, (C₁-C₄)alkoxy(C₁-C₁₀)alkyl and C₂-C₁₀ alkenyl radicals and R1 and R2 are as defined above.

In at least one embodiment, the at least one cyanoacrylate monomer is chosen from ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyanoacrylate, n-octyl 2-cyanoacrylate, allyl 2-cyanoacrylate, methoxypropyl 2-cyanoacrylate and isoamyl cyanoacrylate.

In at least one embodiment, the at least one cyanoacrylate monomer is chosen from monomers b). In at least one further embodiment, the at least one cyanoacrylate monomer is chosen from C₆-C₁₀ alkyl cyanoacrylates.

According to at least one embodiment, the at least one cyanoacrylate monomer is chosen from octyl cyanoacrylates of formula XIII and mixtures thereof:

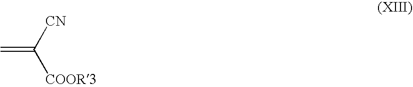

wherein:
R′3 is chosen from:
   —(CH₂)₇—CH₃,
   —CH(CH₃)—(CH₂)₅—CH₃,
   —CH₂—CH(C₂H₅)—(CH₂)₃—CH₃,
   —(CH₂)₅—CH(CH₃)—CH₃, and
   —(CH₂)₄—CH(C₂H₅)—CH₃.

The monomers used in accordance with the disclosure may be covalently bonded to at least one support such as polymers, oligomers or dendrimers. The polymer or the oligomer can be linear, branched, in comb form or in block form. The distribution of the monomers of the disclosure over the polymeric, oligomeric or dendritic structure may be random, in an end position or in the form of blocks.

The at least one cyanoacrylate monomer may be present in an amount ranging from 0.1% to 80% by weight, such as from 0.2% to 60% by weight, and for example, from 0.5% to 50% by weight, relative to the total weight of the cosmetic composition.

In the context of the disclosure, the electrophilic monomers are, for example, capable of polymerizing anionically in the presence of at least one nucleophilic agent. As used herein, the term “anionic polymerization” means the mechanism defined in the publication “Advanced Organic Chemistry,” 3^{rd} Edition by Jerry March, pages 151 to 161.

The nucleophilic agents capable of initiating the anionic polymerization are systems that are known in themselves, capable of generating a carbanion on contact with a nucleophilic agent, such as the hydroxyl ions contained in water. As used herein, the term “carbanion” means the chemical species defined in “Advanced Organic Chemistry,” 3^{rd} Edition by Jerry March, page 141.

The at least one nucleophilic agent can be chosen from molecular compounds, oligomers, dendrimers and polymers that have nucleophilic functional groups. Non-limiting examples of nucleophilic functional groups include the following: R₂N⁻, NH₂⁻, Ph₃C⁻, R₃C⁻, PhNH⁻, pyridine, ArS⁻, R—C≡C⁻, RS⁻, SH⁻, RO⁻, R₂NH, ArO⁻, N₃⁻, OH⁻, ArNH₂, NH₃, I⁻, Br⁻, Cl⁻, RCOO⁻, SCN⁻, ROH, RSH, NCO⁻, CN⁻, NO₃⁻, ClO₄⁻ and H₂O, wherein Ph is a phenyl group; Ar is chosen from aryl groups and R is chosen from C₁-C₁₀ alkyl groups.

The at least one cyanoacrylate monomer can be synthesized according to the known methods described in the art. For example, the at least one cyanoacrylate monomer can be synthesized according to the teaching of U.S. Pat. No. 3,527,224, U.S. Pat. No. 3,591,767, U.S. Pat. No. 3,667,472, U.S. Pat. No. 3,995,641, U.S. Pat. No. 4,035,334 and U.S. Pat. No. 4,650,826.

According to at least one embodiment, the at least one cyanoacrylate monomer can be chosen from monomers capable of polymerizing on keratin fibers under acceptable cosmetic conditions. In at least one embodiment, the polymerization of the at least one cyanoacryate monomer is performed at a temperature of less than or equal to 80° C., which does not prevent the application from being completed by drying under a drying hood, blow-drying or treating with a flat iron or a crimping iron.

The composition of the disclosure comprises at least one nitrogenous alkaline agent of the aqueous ammonia or amine type and also the buffer compositions comprising this type of agent. The at least one nitrogenous alkaline agent can be in its protonated form, it being understood that the pH of the composition conditions the equilibrium between the base and the protonated form. In the context of the disclosure, the term “nitrogenous alkaline agent” is intended to mean these two forms of compounds, for example aqueous ammonia and ammonium.

The at least one nitrogenous alkaline agent can be chosen from aqueous ammonia, amines such as low molecular mass amines, for instance primary, secondary and tertiary alkylamines, primary, secondary and tertiary alkanolamines, for example ethanolamine, alkyl- and hydroxyalkyl-, di- and triamines, such as diaminopropane, diaminopropanol, amino acids, and for example basic amino acids such as lysine and arginine, and also nitrogenous bases of the guanidine type. The at least one nitrogenous alkaline agent may be in the form of a buffer, for example ammoniacal buffers. As used herein, the term “buffer” means a mixture of nitrogenous alkaline agents as defined above, with an acid, for example a weak acid. In at least one embodiment, the buffer is chosen from buffers based on ammonium carbonate, ammonium bicarbonate and ammonium citrate.

According to at least one embodiment, the at least one nitrogenous alkaline agent is chosen from aqueous ammonia and ethanolamine.

In at least one embodiment, the at least one nitrogenous alkaline agent is obtained by means of a nitrogenous alkaline agent donor, by chemical reaction, either spontaneously or in the presence of a catalyst or of an enzymatic system.

In at least one further embodiment, the at least one nitrogenous alkaline agent comprises the use of a mixture of nitrogenous alkaline agents, for example, aqueous ammonia/alkanolamine and aqueous ammonia/amino acid mixtures.

The at least one nitrogenous alkaline agent can be combined with nonnitrogenous alkaline agents such as sodium hydroxide, potassium hydroxide, etc.

The emulsion of the present disclosure may be alkaline, and may range from pH 8 to 13. The pH of this emulsion may be determined according to the final use of this emulsion. For example, if this emulsion is intended for lightening dyeing, the pH of the emulsion may range from 8 to 11, for a final use in hair straightening, the pH may be equal to 13, and for hair straightening or permanent-waving, the pH may range from 6 to 10.

The alkaline agent is, in at least one embodiment, introduced into the aqueous phase.

According to at least one embodiment, the aqueous phase constitutes an amount greater than or equal to 50% by weight of the total weight of the emulsion.

The composition of the disclosure may comprise at least one polymerization inhibitor, for example, anionic and/or free-radical polymerization inhibitors, in order to increase the stability of the composition over time. Non-limiting examples include the following polymerization inhibitors: sulphur dioxide, nitric oxide, boron trifluoride, hydroquinone and derivatives thereof such as hydroquinone monoethyl ether, TBHQ, benzoquinone and derivatives thereof such as duroquinone, catechol and derivatives thereof such as t-butyl catechol and methoxycatechol, anisole and its derivatives such as methoxyanisole or hydroxyanisole, pyrogallol and derivatives thereof, p-methoxyphenol, hydroxybutyltoluene, alkyl sulphates, alkyl sulphites, alkyl sulphones, alkyl sulphoxides, alkyl sulphides, mercaptans, 3-sulphonene, and mixtures thereof, where the alkyl groups in at least one embodiment, are chosen from groups having 1 to 6 carbon atoms.

Inorganic and organic acids can also be used as inhibitors.

Thus, the cosmetic composition according to the disclosure can also comprise at least one inorganic or organic acid, the latter having at least one carboxylic or sulphonic group, having a pKa ranging from 0 to 6, such as phosphoric acid, hydrochloric acid, nitric acid, benzenesulphonic acid, toluenesulphonic acid, sulphuric acid, carbonic acid, hydrofluoric acid, acetic acid, formic acid, propionic acid, benzoic acid, mono-, di- and trichloroacetic acids, salicylic acid, trifluoroacetic acid, octanoic acid, heptanoic acid and hexanoic acid.

In at least one embodiment, the at least one polymerization inhibitor is acetic acid.

The concentration of the at least one polymerization inhibitor in the cosmetic composition of the disclosure can be in a range from 10 ppm to 30% by weight, for example from 10 ppm to 15% by weight, relative to the total weight of the composition.

To modify the anionic polymerization kinetics, it is possible to increase the nucleophilicity of the fiber via chemical conversion of the keratin material.

Examples that may be mentioned include the reduction of the disulphide bridges of which keratin is partly composed, into thiols, before applying the composition of the disclosure. Non-limiting examples of reducing agents for the disulphide bridges of which keratin is partly composed are as follows:
anhydrous sodium thiosulphate,
powdered sodium metabisulphite,
thiourea,
ammonium sulphite,
thioglycolic acid,
thiolactic acid,
ammonium thiolactate,
glyceryl monothioglycolate,
ammonium thioglycolate,
thioglycerol,
2,5-dihydroxybenzoic acid,
diammonium dithioglycolate,
strontium thioglycolate,
calcium thioglycolate,
zinc formosulphoxylate,
isooctyl thioglycolate,
dl-cysteine, and
monoethanolamine thioglycolate.

The water-in-oil emulsion of the disclosure can also comprise at least one adjuvent that is normally used in cosmetics, chosen, for example, from reducing agents, fatty substances, plasticizers, softeners, antifoams, moisturizers, pigments, clays, inorganic fillers, UV-screening agents, inorganic colloids, peptizers, solubilizing agents, fragrances, preserving agents, fixing or non-fixing polymers, polyols, proteins, vitamins, direct or oxidation dyes, pearlescent agents, propellants, and inorganic or organic thickeners, oxyethylenated or nonoxyethylenated waxes, paraffins, C₁₀-C₃₀ fatty acids such as stearic acid or lauric acid, C₁₀-C₃₀ fatty amides such as lauric diethanolamide, C₁₀-C₃₀ fatty alcohol esters such as C₁₀-C₃₀ fatty alcohol benzoates, and mixtures thereof.

According to at least one embodiment, the emulsion comprises at least one cosmetic adjuvant chosen from pigments, nano/micro objects, liquid crystals, oxidizing agents, direct dyes, fluorescent dyes, oxidation dyes, polymers and reducing agents.

The direct dyes may be chosen from, for example, nitrobenzene dyes, azo direct dyes, methine direct dyes, and hydrazone dyes, which may be cationic or noncationic.

The oxidation dyes may be oxidation bases and couplers that are well known in the art. By way of couplers, non-limiting examples include meta-phenylenediamines, meta-aminophenols, meta-diphenols, naphthalene couplers, heterocyclic couplers and addition salts thereof. As oxidation bases, non-limiting examples include para-phenylenediamines, bisphenylalkylenediamines, para-aminophenols, ortho-aminophenols and heterocyclic bases.

The pigments that can be used are known in the art; they are, for example, described in the Encyclopaedia of Chemical Technology by Kirk-Othmer and in the Encyclopaedia of Industrial Chemistry by Ullmann.

These pigments can be in the form of a pigmentary powder or paste. The pigments can, for example, be chosen from white and colored pigments, lakes, pigments with special effects such as pearlescent agents or flakes, and mixtures thereof.

As used herein, the term “polymer” means any of the natural or synthetic polymers that can be used in cosmetics, for example, the polymers obtained by free-radical or anionic polymerization or by polycondensation or by ring opening. These polymers may be linear, branched or star polymers.

In at least one embodiment, the polymer is chosen from natural polymers which may or may not be chemically modified, such as dextrans, celluloses (carboxymethylhydroxypropylcelluloses), guars (carboxymethylhydroxypropylguars), starches, alginates and chitosans.

The pigments and the fillers can be coated with organic or inorganic compounds.

According to at least one embodiment, the pigments and the fillers are coated with fatty substances such as parleam, silicone compounds, fatty acids, or fatty alcohols, such as palmitic acid, palmityl alcohol, stearic acid and stearyl alcohol, and a mixture thereof, where the fatty acids may be in the form of sodium, potassium, magnesium, iron, titanium, zinc or aluminium salts.

The water-in-oil emulsion of the disclosure can be formulated in various galenic forms, such as a lotion, an aerosol foam, a conditioner or a shampoo, a gel, or a wax.

The water-in-oil emulsion of the disclosure may be a cosmetic emulsion. In the context of the disclosure, the term “cosmetic” means an emulsion compatible with keratin materials.

It is possible to prepare several emulsions of the water-in-oil (W/O) type of different compositions and to mix them in varying proportions so as to obtain a new emulsion comprising various cosmetic active agents.

The process of the present disclosure comprises the application of the water-in-oil emulsion described above, to keratin fibers.

The emulsion of the present disclosure may, for example, be used for bleaching keratin fibers. In this case, the emulsion itself may comprise at least one oxidizing agent or the at least one oxidizing agent may be added extemporaneously at the time of use. The oxidizing agents conventionally used are, for example, hydrogen peroxide, urea peroxide, alkali metal perbromates, persalts such as perborates and persulphates, peracids and oxidase enzymes, for example, peroxidases, 2-electron oxidoreductases such as uricases, and 4-electron oxygenases such as laccases.

The emulsion of the present disclosure may be used for oxidation dyeing. In this case, the emulsion may comprise at least one oxidizing agent or at least one oxidation dye precursor. It is also possible to add a composition comprising at least one oxidation dye and/or a composition comprising at least one oxidizing agent to the emulsion of the present disclosure at the time of use.

The emulsion of the present disclosure can be used for direct dyeing. In this case, at least one direct dye may be present in the emulsion of the disclosure or may be added to the emulsion at the time of use. In the case of direct lightening dyeing, the emulsion of the present disclosure may comprise the at least one direct dye and at least one oxidizing agent. According to at least one embodiment, the at least one direct dye and/or the at least one oxidizing agent can be added to the emulsion of the present disclosure at the time of use.

The emulsion of the present disclosure can be used for permanent-waving or thiol hair straightening. In this case, the emulsion can comprise at least one reducing agent such as thioglycolic acid or cysteine. The at least one reducing agent can be added to the emulsion at the time of use.

The emulsion of the present disclosure can be used in the context of alkaline hair straightening. In this case, the emulsion can comprise at least one inorganic base such as sodium hydroxide, lithium hydroxide, ammonium citrate or guanidinium salts. The at least one inorganic base can also be added to the emulsion at the time of use.

The emulsion of the disclosure can be used in hairstyling, care or hygiene compositions. Thickeners such as carbopols, polymers such as anionic polymers, for instance polyacrylics or methacrylics, anionic surfactants, cationic surfactants, or amphoteric or non-ionic surfactants are added thereto either directly, or in the form of a mixture, at the time of use.

According to the process of the disclosure, the ready-to-use emulsion or composition resulting from the mixing of the emulsion with a cosmetic composition as described above can be applied to dry hair or hair that has been wetted or shampooed beforehand. The hair can then be dried (for example, by hairdryer, hood or iron). A final shampooing procedure can optionally be carried out.

According to at least one embodiment, the hair is pretreated with at least one nucleophilic agent. The hair can be dried. The emulsion of the disclosure can then be applied to the hair. The hair is dried (for example, by hairdryer, hood or iron). A final shampooing procedure can optionally be carried out.

According to at least one further embodiment, the hair is pretreated with at least one cosmetic additive as defined above. The hair can be dried, and the emulsion of the disclosure is then applied to the hair. The hair is dried (for example, by hairdryer, hood or iron). A final shampooing procedure can optionally be carried out.

According to at least one embodiment, the process of the disclosure can comprises combining the application of the emulsion of the disclosure with other treatments on the fiber, as described above.

Thus, according to at least one embodiment, it is possible to apply, as a pretreatment, a permanent-wave reducing agent or permanent-waving or oxidation dyeing or bleaching or shampooing or a hairstyling product or alkaline hair straightening, as a pretreatment which is followed by the application of one of the processes described above.

In at least one embodiment, the emulsion of the disclosure is applied as a pretreatment in order to protect the keratin fiber before the application of treatments known to be capable of degrading the hair. It is then possible to optionally remove the makeup from the fiber.

Thus, after having applied the composition of the disclosure according to one of the processes already described, a permanent-wave reducing agent or permanent-waving or oxidation dyeing or bleaching or alkaline hair straightening may be applied.

The application of the emulsion of the disclosure can also be carried out after oxidation or direct dyeing, or a mixture of these dyes, in order to protect the brightness of the color against shampooing.

A subject of the present disclosure is also a kit comprising at least one first composition comprising an emulsion as described above and at least one second composition comprising at least one nucleophilic agent.

A subject of the disclosure is also a kit comprising at least one first composition comprising at least one cyanoacrylate monomer and, optionally, at least one anionic and/or free-radical polymerization inhibitor and/or an acid, and at least one second composition comprising a water-in-oil emulsion and, optionally, an organic acid.

According to at least one embodiment, at least one first composition comprises a water-in-oil emulsion which comprises at least one aqueous phase, at least one oily phase, at least one surfactant and at least one cyanoacrylate monomer, and optionally at least one acid, and at least one second composition comprises at least one nitrogenous alkaline agent.

According to at least one embodiment, the at least one first composition comprises the at least one cyanoacrylate monomer and, optionally, an acid and the at least one second composition comprises at least one aqueous phase, at least one oily phase, at least one surfactant and at least one nitrogenous alkaline agent in the form of the water-in-oil emulsion of the disclosure (aqueous phase, oily phase, surfactant and nitrogenous alkaline agent).

According to at least one further embodiment, a first composition comprises at least one cyanoacrylate monomer and, optionally, at least one acid, a second composition comprises at least one nitrogenous alkaline agent and a third composition is in the form of a water-in-oil emulsion comprising at least one aqueous phase, at least one oily phase and at least one surfactant.

The process of the disclosure can be carried out several times in order to perform multiple applications of the emulsion of the disclosure so as to obtain a superimposition of layers in order to attain specific coating properties in terms of chemical nature, mechanical strength, thickness, appearance and feel.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in its respective testing measurement.

The examples that follow are intended to illustrate the present disclosure without, however, being limiting in nature.

### EXAMPLES

### Example 1

The following table illustrates examples of emulsions according to the present disclosure in which 2% aqueous ammonia was used as the at least one nitrogenous alkaline agent and was introduced into the aqueous phase. For each of these emulsions, the smell of ammonia was virtually eliminated.

| | Silicone O/W emulsion | | | | | | | |
|---|---|---|---|---|---|---|---|---|
| | Ex. 1.1 | Ex. 1.2 | Ex. 1.3 | Ex. 1.4 | Ex. 1.5 | Ex. 1.6 | Ex. 1.7 | Ex. 1.8 |
| 1-Methylheptyl cyanoacrylate | 9.10% | 4.80% | 9.10% | 4.80% | 4.20% | 10% | 5% | 10% |
| Cyclopentasiloxane | 13.15% | 6.90% | 13.15% | 6.90% | 7% | 10% | 7% | 5% |
| PEG/PG-18/18 dimethicone | 0.45% | 0.20% | 0.45% | 0.24% | 0.50% | 1% | 0.50% | 0.50% |
| Acetic acid | 27.30% | 14.30% | 9.10% | 4.76% | 2.70% | 1.50% | 1.50% | 1.50% |
| Water | 50% | 74% | 68% | 83% | 86% | 78% | 86% | 83% |
| Fatty phase | 22.70% | 11.90% | 22.70% | 11.90% | 11.70% | 21% | 12.50% | 15.50% |
| Aqueous phase | 77.30% | 88.10% | 77.30% | 88.10% | 88.30% | 79% | 87.50% | 84.50% |

Non-silcone W/O emulsion

| | Ex. 1.9 | Ex. 1.10 |
|---|---|---|
| 1-Methylheptyl cyanoacrylate | 5% | 5% |
| Mineral oil/paraffinium liquidium | 7% | 5% |
| Olea europaea (olive) fruit oil/olea europaea | — | 2% |
| Hydroxyethyldiethonium polyisobutenyl | 1% | 1% |
| triethylaminosuccinate | | |
| Acetic acid | 3% | 3% |
| Water | 84% | 84% |
| Fatty phase | 13% | 13% |
| Aqueous phase | 87% | 87% |

### Example 2

### Limiting the Odor of Ammonia in a Lightening Hair Formula

A water-in-oil emulsion “E” at 90 g % was prepared as follows:
Fatty phase: 2.8 g of cyclopentasiloxane (100% AM) and 8.33 g of a W/O emulsifier consisting of oxyethylenated oxypropylenated (18 OE/18 OP) polydimethylsiloxane+cyclopentadimethylsiloxane+water (10/88/2) were introduced into a beaker A.
Aqueous phase: 10.2 g of aqueous ammonia (solution containing 20.5 g % of ammonia) and 68.67 g of water were introduced into a beaker B.
The aqueous phase B was gradually introduced into the fatty phase A with stirring in a Rayneri mixer (900/1400 rpm).

10 g % of 2-octyl cyanoacrylate were added to the inverse emulsion “E”. 5 g of the composition obtained were mixed with 5 g of 40-volume oxidant (12 g % aqueous hydrogen peroxide).

The mixture “C1” thus obtained did not smell of ammonia.

In parallel, 5 g of the inverse emulsion “E” were mixed with 5 g of 40-volume oxidant (12 g % aqueous hydrogen peroxide). The mixture obtained, “C2”, smelled strongly of ammonia.

The lightening capacity of the mixture C1 was compared with that of the mixture C2. For this, each mixture was applied to a lock of 1 g of chestnut brown hair (tone depth=4). The waiting period was 30 min at ambient temperature. Finally, the lock was washed and subjected to shampooing.

The level of lightening of the locks was evaluated in the L* a* b* system, by means of a Minolta® CM 2002 spectrophotometer (D65 illuminant). In this L* a* b* system, L* represents the intensity of the color, a* indicates the green/red color axis and b* the blue/yellow color axis. The lower the value of L, the deeper or much more intense the color. As the value of a* increases, the shade becomes more red, and as the value of b* increases, the shade becomes more blue. The measurement of L*a*b* was carried on the locks before and after lightening. The level of lightening was measured by the parameter ΔE according to the following equation:
ΔE=\sqrt{(L₁−L₀)²+(a₁*−a₀*)²+(b₁*−b₀*)²}

L₀, a₀* and b₀* define the calorimetric values before lightening and L₁, a₁* and b₁* define the calorimetric values after lightening.

As indicated in the table below, the lock treated with the mixture C1 had a level of lightening comparable to that of the lock treated with the mixture C2.

| | L* | a* | b* | ΔE*ab |
|---|---|---|---|---|
| Natural hair tone depth 4 | 21.61 | 2.68 | 3.06 | |
| Natural hair tone depth 4, | 25.66 | 6.25 | 8.52 | 7.68 |
| treated with the mixture C1 | | | | |
| Natural hair tone depth 4, | 26.51 | 6.34 | 8.21 | 8.00 |
| treated with the mixture C2 | | | | |

What is claimed is:

1. A water-in-oil emulsion for the treatment of keratin fibers, comprising at least one surfactant; an aqueous phase comprising at least one nitrogenous alkaline agent and an oily phase comprising at least one cyanoacrylate monomer that can polymerize anionically in the presence of a nucleophilic agent.

2. The water-in-oil emulsion according to claim 1, wherein the oily phase comprises at least one liquid organic solvent.

3. The water-in-oil emulsion according to claim 2, wherein the at least one organic solvent is chosen from aromatic alcohols; liquid fatty alcohols, polyols; volatile silicones; mineral, organic and plant oils; alkanes; liquid fatty acids and liquid fatty esters.

4. The water-in-oil emulsion according to claim 3, wherein the at least one organic solvent comprises at least one volatile silicone.

5. The water-in-oil emulsion according to claim 1, wherein the at least one cyanoacrylate monomer is chosen from the monomers of formula (I): 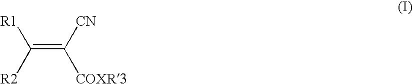
wherein:
X is chosen from NH, S and O,
R1 and R2 are chosen from, independently of one another, sparingly electron-withdrawing and non-electron-withdrawing groups
R′3 is chosen from a hydrogen atom and saturated and unsaturated, linear, branched and cyclic hydrocarbon-based groups, optionally containing at least one entity chosen from nitrogen, oxygen and sulphur atoms, and optionally substituted with at least one entity chosen from —OR′, —COOR′, —COR′, —SH, —SR′, —OH, and halogens, and polymer residues that may be obtained by free-radical polymerization, by polycondensation and by ring opening, wherein R′ is chosen from C₁-C₁₀ alkyl groups.

6. The water-in-oil emulsion according to claim 5, wherein:
R1 and R2 are chosen from:
hydrogen atoms,
saturated and unsaturated, linear, branched and cyclic hydrocarbon-based groups and optionally containing at least one entity chosen from nitrogen, oxygen and sulphur atoms, and optionally substituted with at least one entity chosen from —OR, —COOR, —COR, —SH, —SR, —OH, and halogens,
modified and unmodified polyorganosiloxane residues, and
polyoxyalkylene groups,
wherein R is chosen from saturated and unsaturated, linear, branched and cyclic hydrocarbon-based groups and optionally containing at least one entity chosen from nitrogen, oxygen and sulphur atoms, and optionally substituted with at least one entity chosen from —OR′, —COOR′, —COR′, —SH, —SR′, —OH, and halogens, and polymer residues that may be obtained by free-radical polymerization, by polycondensation and by ring opening, wherein R′ is chosen from C₁-C₁₀ alkyl groups,

7. The water-in-oil emulsion according to claim 6, wherein:
R1 and R2 are chosen from saturated and unsaturated, linear, branched and cyclic hydrocarbon-based groups containing from 1 to 20 carbon atoms and optionally containing at least one entity chosen from nitrogen, oxygen and sulphur atoms, and optionally substituted with at least one entity chosen from —OR, —COOR, —COR, —SH, —SR, —OH, and halogens, wherein R is chosen from saturated and unsaturated, linear, branched and cyclic hydrocarbon-based group and optionally containing at least one entity chosen from nitrogen, oxygen and sulphur atoms, and optionally substituted with at least one entity chosen from —OR′, —COOR′, —COR′, —SH, —SR′, —OH, and halogens, and polymer residues that may be obtained by free-radical polymerization, by polycondensation and by ring opening, wherein R′ is chosen from C₁-C₁₀ alkyl groups.

8. The water-in-oil emulsion according to claim 6, wherein:
R and R′3 are chosen from saturated and unsaturated, linear, branched and cyclic hydrocarbon-based groups containing from 1 to 20 carbon atoms and optionally containing at least one entity chosen from nitrogen, oxygen and sulphur atoms, and optionally substituted with at least one entity chosen from —OR′, —COOR′, —COR′, —SH, —SR′, —OH, and halogens, and polymer residues that may be obtained by free-radical polymerization, by polycondensation and by ring opening, wherein R′ is chosen from C₁-C₁₀ alkyl groups.

9. The water-in-oil emulsion according to claim 5, wherein the at least one cyanoacrylate monomer is chosen from the monomers of formula (IV): 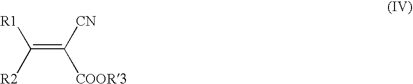
wherein R′3 is chosen from C₁-C₁₀ alkyl, C₂-C₁₀ alkenyl and (C₁-C₄)alkoxy(C₁-C₁₀)alkyl radicals.

10. The water-in-oil emulsion according to claim 9, wherein the at least one cyanoacrylate monomer is chosen from ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyanoacrylate, n-octyl 2-cyanoacrylate, isoamyl cyanoacrylate, allyl 2-cyanoacrylate and methoxypropyl 2-cyanoacrylate.

11. The water-in-oil emulsion according to claim 9, wherein the at least one cyanoacrylate monomer is chosen from C₆-C₁₀ alkyl cyanoacrylates.

12. The water-in-oil emulsion according to claim 9, wherein the at least one cyanoacrylate monomer is chosen from the octyl cyanoacrylate monomers of formula (V): 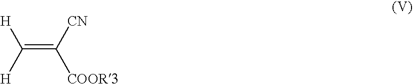
wherein R′3 is chosen from:
—(CH₂)₇—CH₃,
—CH(CH₃)—(CH₂)₅—CH₃,
—CH₂—CH(C₂H₅)—(CH₂)₃—CH₃,
—(CH₂)₅—CH(CH₃)—CH₃, and
—(CH₂)₄—CH(C₂H₅)—CH₃.

13. The water-in-oil emulsion according to claim 1, wherein the at least one cyanoacrylate monomer is present in an amount ranging from 0.1% to 80% by weight, relative to the total weight of emulsion.

14. The water-in-oil emulsion according to claim 13, wherein the at least one cyanoacrylate monomer is present in an amount ranging from 0.5% to 50% by weight, relative to the total weight of emulsion.

15. The water-in-oil emulsion according to claim 1, wherein the at least one nitrogenous alkaline agent is chosen from aqueous ammonia, alkylamines, alkanolamines, alkyl- and hydroxyalkyl-, di- and triamines, and nitrogenous bases.

16. The water-in-oil emulsion according to claim 1, wherein the at least one nitrogenous alkaline agent is chosen from aqueous ammonia and ethanolamine.

17. The water-in-oil emulsion according to claim 1, wherein the at least one nitrogenous alkaline agent is produced from at least one nitrogenous alkaline agent donor, via chemical reaction, either spontaneously or in the presence of a catalyst or of an enzymatic system.

18. The water-in-oil emulsion according to claim 1, wherein the water-in-oil emulsion further comprises at least one polymerization inhibitor chosen from sulphur dioxide, nitric oxide, boron trifluoride, hydroquinone and derivatives thereof, benzoquinone and derivatives thereof, catechol and derivatives thereof, anisole and derivatives thereof, pyrogallol and derivatives thereof, p-methoxyphenol, hydroxybutyltoluene, alkyl sulphates, alkyl sulphites, alkyl sulphones, alkyl sulphoxides, alkyl sulphides, mercaptans, 3-sulphonene, and organic acids.

19. The water-in-oil emulsion according to claim 18, wherein the at least one polymerization inhibitor is acetic acid.

20. The water-in-oil emulsion according to claim 1, wherein the at least one surfactant is chosen from silicone surfactants.

21. The water-in-oil emulsion according to claim 1, wherein the at least one surfactant is chosen from non-silicone surfactants.

22. The water-in-oil emulsion according to claim 1, wherein the at least one surfactant is present in an amount ranging from 0.1% to 30% by weight of emulsion.

23. The water-in-oil emulsion according to claim 1, wherein the aqueous phase is present in an amount greater than or equal to 50% by weight of the total weight of the emulsion.

24. The water-in-oil emulsion according to claim 1, wherein the at least one surfactant is chosen from non-ionic surfactants and has an HLB of less than 10.

25. A process for treating keratin materials, said process comprising applying to said keratin materials a water-in-oil emulsion comprising at least one surfactant; an aqueous phase comprising at least one nitrogenous alkaline agent and an oily phase comprising at least one cyanoacrylate monomer that can polymerize anionically in the presence of a nucleophilic agent.

26. The process according to claim 25, further comprising pretreating the keratin materials with at least one nucleophilic agent.

27. A kit for treating keratin fibers, comprising at least one first composition which comprises a water-in-oil emulsion for the treatment of keratin fibers, comprising at least one surfactant; an aqueous phase comprising at least one nitrogenous alkaline agent and an oily phase comprising at least one cyanoacrylate monomer that can polymerize anionically in the presence of a nucleophilic agent, and a second composition comprising at least one nucleophilic agent.

28. A kit for treating keratin fibers, comprising at least one first composition comprising at least one cyanoacrylate monomer that can polymerize anionically in the presence of a nucleophilic agent, and, optionally comprising at least one anionic and/or free-radical polymerization inhibitor and/or acid, and at least one second composition comprising a water-in-oil emulsion comprising an aqueous phase comprising at least one nitrogenous alkaline agent and an oily phase, at least one surfactant and, optionally, at least one organic acid.
